(19) United States
(12) Patent Application Publication
(10) Pub. No.: US 2026/0284397 A1
(43) Pub. Date: Sep. 24, 2026

(54) USING ACTIVITY THRESHOLDS TO DRIVE ADAPTIVE PAIN THERAPY

(71) Applicant: Boston Scientific Neuromodulation Corporation, Valencia, CA (US)

(72) Inventor: Lisa Denise Moore, Glendale, CA (US)

(73) Assignee: Boston Scientific Neuromodulation Corporation, Valencia, CA (US)

(21) Appl. No.: 19/575,449

(22) Filed: Mar. 23, 2026

Related U.S. Application Data
(60) Provisional application No. 63/776,654, filed on Mar. 24, 2025.

(51) Int. Cl. A61N 1/36; A61N 1/05; A61B 5/11

(52) U.S. Cl. CPC A61N 1/36071; A61N 1/0551; A61N 1/36132; A61N 1/36139; A61N 1/3615; A61B 5/1118

(57) ABSTRACT

A method and system for treating pain using an implanted spinal cord stimulation system monitors patient activity levels over a baseline period to determine a baseline activity level. High and low activity thresholds are set relative to the baseline to define activity ranges. The system monitors patient activity during a monitoring period to determine if measured activity falls within the high or low activity ranges. When activity exceeds the high threshold, a first stimulation bolus is scheduled. When activity falls below the low threshold, a second stimulation bolus is scheduled. The system can track effectiveness of the stimulation and modify parameters based on monitored responses.

### CROSS REFERENCE TO RELATED APPLICATIONS

This application claims the benefit of U.S. Provisional Patent Application Serial No. 63/776,654 filed on March 24, 2025, the disclosure of which is incorporated herein by reference.

### BACKGROUND

Spinal cord stimulation (SCS) systems may deliver electrical pulses to nervous tissue of or near the spinal cord to treat a variety of disorders, such as chronic pain. Although the pain that a patient experiences may improve overall, there still exist fluctuations in pain on a shorter timescale. Adjustments in the stimulation parameters of the electrical pulses may compensate for fluctuations, however adjustment of the stimulation parameters may require a visit to a physician, delaying pain relief.

Multiple strategies have been developed to address these fluctuations; however, these existing strategies merely provide a repeatable and tonic pattern of changes that are non-adaptive to the patient or overly rely on calibration and detection accuracy.

### OVERVIEW

The present inventors have recognized, among other things, that a problem to be solved is to provide a SCS system to deliver stimulation parameters that are adaptive to the patient's activity level.

A first illustrative and non-limiting example takes the form of a method for treating a patient having a pain disorder with an implanted spinal cord stimulation system, the method comprising: monitoring an activity level of the patient during a baseline period; determining a baseline activity level for the patient over the baseline period; setting a high activity threshold and a low activity threshold relative to the baseline activity level to define: a high activity range above the high activity threshold, and a low activity range below the low activity threshold; monitoring the activity level of the patient during a monitoring period, wherein the monitoring period is at least one hour; determining whether a measured patient activity level falls within one of: the high activity range and the low activity range; scheduling a first bolus of stimulation from the implanted spinal cord stimulation system in response to a determination that the measured patient activity level falls within the high activity range; and scheduling a second bolus of stimulation from the implanted spinal cord stimulation system in response to a determination that the measured patient activity level falls within the low activity range.

Additionally or alternatively, monitoring the activity level of the patient comprises receiving activity data from at least one of an accelerometer, a cardio fitness monitor, a fitness tracker application, or patient interaction with a remote control device.

Additionally or alternatively, determining the baseline activity level comprises: calculating a daily average activity level over at least one week; and determining a cumulative activity level for at least one time window within each day.

Additionally or alternatively, the method also includes periodically recalculating the baseline activity level based on the measured activity levels; and adjusting the high activity threshold and low activity threshold based on the recalculated baseline activity level.

Additionally or alternatively, scheduling the first bolus of stimulation further comprises: scheduling a preventative stimulation dose after a delay period, wherein the delay period is modified based on a detected change in activity level; delivering the preventative stimulation dose at a modified intensity relative to a pain bolus; and delivering the preventative stimulation dose for a predetermined duration, wherein the predetermined duration is adjustable in response to the measured patient activity level.

Additionally or alternatively, scheduling the second bolus of stimulation further comprises: increasing a stimulation dose for a first predetermined time period; evaluating the activity level of the patient near the end of the first predetermined time period; and extending, reducing, or terminating the increased stimulation dose based on the evaluated patient activity.

Additionally or alternatively, the method also includes monitoring changes in measured patient activity level following the first bolus of stimulation and the second bolus of stimulation to determine an effectiveness of each bolus; determining a modification to stimulation duration and stimulation dose level for each of the first bolus of stimulation and the second bolus of stimulation based on the determined effectiveness of each bolus; automatically modifying the stimulation duration and the stimulation dose level of a subsequent bolus based on monitored patient responses; and storing the modified stimulation duration and the modified stimulation dose level for future boluses of stimulation.

Additionally or alternatively, monitoring the activity level of the patient during a monitoring period comprises calculating a daily average activity level during the course of a day.

Additionally or alternatively, monitoring the activity level of the patient during a monitoring period comprises determining a cumulative activity level for at least one time window within each day, the at least one time window being at least one hour long.

Another illustrative and non-limiting example takes the form of a method for managing pain therapy via an implanted spinal cord stimulation system, comprising: determining a baseline activity level by measuring a patient activity level over a baseline period; measuring an activity level of a patient over defined time periods to determine a cumulative activity level; setting a first threshold and a second threshold relative to the baseline activity level to define: a first activity range above the first threshold, a second activity range below the second threshold; comparing the cumulative activity level to the first threshold and the second threshold to determine whether the cumulative activity level falls within one of: the high activity range and the low activity range; performing a first response including scheduling a first bolus of stimulation from the implanted spinal cord stimulation system in response to a determination that the measured patient activity level falls within the high activity range; performing a second response including scheduling a second bolus of stimulation from the implanted spinal cord stimulation system in response to a determination that the measured patient activity level falls within the low activity range; monitoring an effectiveness of the first bolus of stimulation or the second bolus of stimulation; and modifying at least one stimulation parameter based on monitored effectiveness.

Additionally or alternatively, performing the first response comprises: applying a first delay before initiating the first bolus of stimulation in response to the measured patient activity level exceeding the first threshold, and delivering the first bolus of stimulation after the first delay; wherein the first delay is modified based on a detected change in patient activity level.

Additionally or alternatively, performing the second response comprises: increasing a stimulation dose for a first predetermined time period; evaluating the patient activity level near the end of the first predetermined time period; and extending, reducing, or terminating the increased stimulation dose based on the evaluated patient activity level.

Additionally or alternatively the cumulative activity level is reset at least daily.

Additionally or alternatively, measuring the patient activity level comprises: tracking at least one of: movement, heart rate, sweat, oxygen level, or respiration; tracking measured patient activity levels over time; and determining the cumulative activity level over a time period of at least one hour.

Additionally or alternatively, the method further includes setting a maximum duration on modifications to the stimulation parameter; prompting review when the maximum duration is reached; and recommending program modifications based on the monitored effectiveness.

Additionally or alternatively, monitoring the effectiveness comprises: monitoring a change in the measured patient activity level following the first bolus of stimulation or the second bolus of stimulation; determining, based on the monitored change, whether the measured patient activity level returns to the baseline activity level; and adjusting the stimulation parameter based on the monitored change.

Additionally or alternatively, the method also includes receiving patient feedback on the first bolus of stimulation or the second bolus of stimulation; modifying the stimulation parameter based on the patient feedback storing the modified stimulation parameter for future use.

Additionally or alternatively, the method also includes setting a maximum stimulation threshold and a minimum stimulation threshold during an initial programming; automatically recommending stimulation parameters based on perception information collected during the initial programming; and modifying the stimulation parameters based on collected perception information within the maximum stimulation threshold and the minimum stimulation threshold.

Additionally or alternatively, the method also includes tracking the measured patient activity level; updating the baseline activity level, the high-activity threshold, and the low-activity threshold based on a tracked patient activity level; and adapting the stimulation parameter based on the updated baseline activity level.

Another illustrative and non-limiting example takes the form of a system for providing adaptive pain therapy, comprising: an implantable pulse generator configured to deliver spinal cord stimulation; at least one activity sensor configured to monitor an activity level of a patient; a processor with a communication circuit for communicating with the implantable pulse generator, wherein the processor is configured to: determine a baseline activity level during a baseline period; set a high activity threshold and a low activity threshold relative to the baseline activity level; measure the patient activity level during a monitoring period of at least one hour; schedule a first bolus of stimulation from the implantable pulse generator in response to the measured activity level exceeding the high activity threshold; and schedule a second bolus of stimulation from the implantable pulse generator in response to the measured activity level falling below the low activity threshold.

Additionally or alternatively, the activity sensor is configured to receive activity data from at least one of an accelerometer, a cardio fitness monitor, a fitness tracker application, or patient interaction with a remote control device.

Additionally or alternatively, the processor is further configured to: calculate a daily average activity level over at least one week; and determine a cumulative activity level for at least one time window within each day.

Additionally or alternatively, wherein the processor is further configured to: periodically recalculate the baseline activity level based on the measured activity levels; and adjust the high activity threshold and low activity threshold based on the recalculated baseline activity level.

Additionally or alternatively, the processor is configured to: schedule a preventative stimulation dose after a delay period, wherein the delay period is modified based on a detected change in activity level; issue an instruction, via the communication circuit, to the implantable pulse generator to: deliver the preventative stimulation dose at a modified intensity relative to a pain bolus; and deliver the preventative stimulation dose for a predetermined duration, wherein the predetermined duration is adjustable in response to the measured patient activity level.

Additionally or alternatively, in scheduling the second bolus of stimulation, the processor is configured to: determine a stimulation dose increase for a first predetermined time period; evaluate the activity level of the patient near the end of the first predetermined time period; and issue an instruction, via the communication circuit, to the implantable pulse generator to extend, reduce, or terminate the stimulation dose increase based on the evaluated patient activity level.

Additionally or alternatively, the processor is configured to: monitor changes in measured patient activity level following the first bolus of stimulation and the second bolus of stimulation to determine an effectiveness of each bolus; determine a modification to stimulation duration and stimulation dose level for each of the first bolus of stimulation and the second bolus of stimulation based on the determined effectiveness of each bolus; issue an instruction, via the communication circuit, to the implantable pulse generator to automatically modify the stimulation duration and the stimulation dose level of a subsequent bolus based on monitored patient responses; and store the modified stimulation duration and the modified stimulation dose level for future boluses of stimulation.

Additionally or alternatively, the processor is configured to calculate a daily average activity level during the course of a day.

Additionally or alternatively, the processor is configured to determine a cumulative activity level for at least one time window within each day, the at least one time window being at least one hour long.

Additionally or alternatively, the processor is configured to: set a maximum duration on modifications to the stimulation parameter; prompt review when the maximum duration is reached; and recommend program modifications based on the monitored effectiveness.

Additionally or alternatively, the processor is configured to: receive patient feedback on the first bolus of stimulation or the second bolus of stimulation; modify the stimulation parameter based on the patient feedback; and store the modified stimulation parameter for future use.

Additionally or alternatively, the processor is configured to: set a maximum stimulation threshold and a minimum stimulation threshold during an initial programming; automatically recommend stimulation parameters based on perception information collected during the initial programming; and modify the stimulation parameters based on collected perception information within the maximum stimulation threshold and the minimum stimulation threshold.

Additionally or alternatively, the processor is configured to: track the measured patient activity level; update the baseline activity level, the high-activity threshold, and the low-activity threshold based on a tracked patient activity level; and adapt the stimulation parameter based on the updated baseline activity level.

Additionally or alternatively, the processor is configured to: detect a repeated pattern of the measured activity level exceeding the high activity threshold or falling below the activity threshold; modify the stimulation parameters based on detected patterns; and generate a notification for clinical review when the repeated patterns of threshold crossings are detected.

Additionally or alternatively, the system further comprises a patient notification system configured to alert the patient of an automatic stimulation parameter adjustment; and a patient override interface configured to allow the patient to modify or cancel the automatic stimulation parameter adjustment.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative SCS system.

FIG. 2 shows an illustrative method of determining baseline activity level of a patient in block flow form.

FIGS. 3A-C show an illustrative method of scheduling and delivering a bolus of stimulation in block flow form.

FIG. 4 shows factors contributing to the modification of stimulation parameters.

FIG. 5 shows an illustrative method relating to maximum durations on modifications to the stimulation parameters.

FIGS. 6A-B shows an illustrative patient notification system.

### DETAILED DESCRIPTION

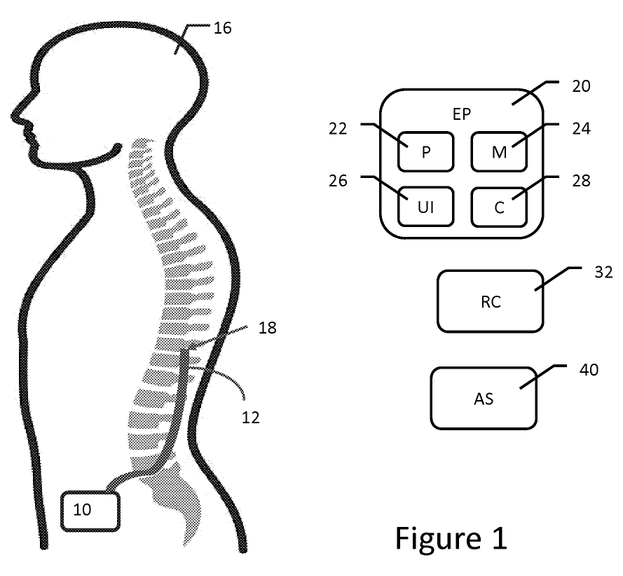

FIG. 1 shows a an illustrative SCS system (herein referred to as “the system”) implanted in a patient 16. The system includes an implantable pulse generator (IPG) 10, shown implanted in the lower abdomen region of the patient 16. The IPG 10 is coupled to a lead 12 which extends subcutaneously to the spinal cord and then threaded toward or near a stimulation target site 18 of the patient 16. The lead 12 includes a plurality of electrodes (not illustrated) to deliver electrical stimulation to the stimulation target site 18 in the spinal cord of the patient 16. The stimulation target site 18 in the spinal cord of the patient 16 may include, but is not limited to, the epidural space, the subdural space, caudal space, intrathecal space, or in some cases, the peripheral nerves to the spinal cord. The stimulation target site may be selected dependent on the needs of the procedure and patient 16 among other considerations.

The IPG 10 may include separate circuits, sometimes referred to as operational circuitry, including a microcontroller (which may also be implemented as part of a microprocessor if desired), which controls operations of the IPG 10 at a high level. The IPG 10 can include a power source, typically a battery (rechargeable or primary cell, as desired), though some systems may be adapted to operate without a battery by receiving power inductively or through other link (such as radiofrequency) and issuing therapy using the received power without long-term storage. The microcontroller may include memory for storing operational instructions in a non-transitory media, such as a Flash memory, RAM, ROM, etc.

The IPG 10 also includes stimulation circuitry. At a high level, the stimulation circuitry may include a plurality of current sources and current sinks (for a current-controlled system; a plurality of voltage sources may be used in voltage-controlled systems instead), and control circuitry including for example one or more analog ASICs, as well as switch arrays that implement steering instructions and/or electrode selections. US Patent 10,716,932 provides illustrative examples and details for both current and planned future implementations of the stimulation circuitry and is incorporated herein by reference. As noted, other designs for stimulation circuitry may be used.

The IPG 10 may include a conductive outer housing that can serve as a return electrode or indifferent electrode during therapy delivery, as desired. A header provides feedthrough circuitry allowing the IPG 10 to couple to a lead 12, with separate electrical connections to each of the electrodes of the lead 12. A lead 12 may include, for example and without limitation, 4, 8, 16 or even 32 electrodes, and may be in the form any of a linear lead, a paddle lead, or a directional or segmented electrode lead, as desired. Any number of leads 12 may be included, such as one, two or even four (or more) leads. The IPG header and housing provide a hermetic sealed environment for the operational circuitry. In some examples, a plurality of programs can be set for therapy delivery by the IPG 10. Each program may operate according to a schedule and individual program stimulation parameters.

The illustrative system described below includes various external devices. An external clinician programmer (CP) 20 may be used to determine/select therapy programs. The CP 20 can be used by a physician to manipulate the outputs of the IPG 10. For example, the CP 20 can be used by the physician to define a therapy regimen or program for application to the patient. Multiple programs may be facilitated and stored by the IPG 10; in some examples, a patient remote control (RC) 32 may store the programs to be used.

The CP 20 may be, for example, and without limitation, a computer such as a laptop or tablet computer. The CP 20 therefore includes a microcontroller and/or microprocessor, shown as processor 22 and associated memory 24. The memory 24 may take any suitable form (RAM, ROM, Flash, etc.), and stores machine readable instructions allowing the processor 22 to perform the methods disclosed herein.

To the extent Bluetooth is used as a communications protocol, the RF circuitry may be included in the device as a communications circuitry 28 located internal to the CP 20. If some other communications technology (inductive or Medradio) is used, or if range is limited by the IPG for example, the communications circuit 28 may be provided via a wand having specialized circuitry (for Bluetooth, Medradio, or inductive telemetry) therein that couples, for example, to a USB port on the CP 20. The CP 20 may include a user interface 26, such as a screen or touchscreen, keyboard, mouse, trackball, etc. allowing the user to provide instructions and make choices. The RC may take the form of a dedicated device, a locked off-the shelf device (such as a smartphone) or may be a multi-use device such as a smartphone running an app. In some examples, the IPG 10 may include a similar architecture of a controller or processor 22, memory 24, and communications circuitry 28.

The CP 20 may be used to determine stimulation parameters. Stimulation parameters may include amplitude of stimulation pulses, frequency or repetition rate of stimulation pulses, pulse width of stimulation pulses, and more complex parameters such as burst therapy, as are known in the art. Biphasic square waves are commonly used, though nothing in the present invention is limited to biphasic square waves, and ramped, triangular, sinusoidal, monophasic and other stimulation types may be used as desired. The CP 20 may be used by a physician, or at the direction of a physician, to obtain data from and provide instructions to the IPG 10 via suitable communications protocols such as Bluetooth or MedRadio or other wireless communications standards, non-standard or proprietary RF, optical and/or via other modalities such as inductive telemetry.

Similarly, the IPG 10 may also include a corresponding communications circuit to communicate with the CP 20. In addition to basic programming and status updates, in which the CP 20 and IPG 10 exchange messages related to therapy parameters, errors in the IPG, battery status, lead status, and other known data exchanges, the communication link may also be used by the IPG to send data, such as activity data, to the CP 20. Activity data may be received at the CP 20 and/or RC 32 from various sources, including activity sensors 40, for example, a heart rate monitor, wearable fitness or movement trackers, a cardio fitness monitoring/tracking smartphone application, accelerometer readings, and patient interaction data from the RC 32. Activity data can be assessed and/or used by any of the CP 20, IPG 10, and/or RC 32, and/or cloud resources in communication with any of these devices, for use in the method which are described below.

Conditions to be treated may include chronic pain conditions, such as failed back surgery syndrome, complex regional pain syndrome, and other chronic and intractable pain; movement-related disorders, such as Parkinson's disease, dystonia, and essential tremor; and psychological and neurological disorders, such as depression, anxiety disorders, addiction, and mood disorders; and other conditions such as, spinal cord injury/degeneration, urinary incontinence, subluxation, sleep apnea, or overactive bladder/bladder voiding problems.

The disclosed SCS system allows for automatic modification of stimulation parameters by continuously monitoring patient activity data to determine when therapeutic intervention is needed and assist with preventing overtreatment and/or undertreatment with SCS.

The figures and explanations herein focus primarily on use of the present invention for SCS purposes. That said, the processes that are disclosed may also be used in other neural therapy systems, including any of Vagus nerve stimulation (VNS), peripheral nerve stimulation (PNS), deep brain stimulation (DBS), occipital nerve therapy, sacral nerve therapy, etc.

At an initial programming session, the system first establishes a baseline activity level by measuring patient activity over an extended period and sets both a high and a low activity threshold relative to this baseline. When patient activity exceeds the high threshold, the system schedules a preventative stimulation dose after a set delay period. Conversely, when activity falls below the low threshold, the system immediately initiates a pain bolus for a predetermined period. The system response may be one sided (configured to treat only when patient activity exceeds the high threshold, or configured to treat only when patient activity is below the low threshold), or two sided (treatment in response to either the high or low threshold being crossed). If a one sided response is set, then only one of the thresholds would need to be configured.

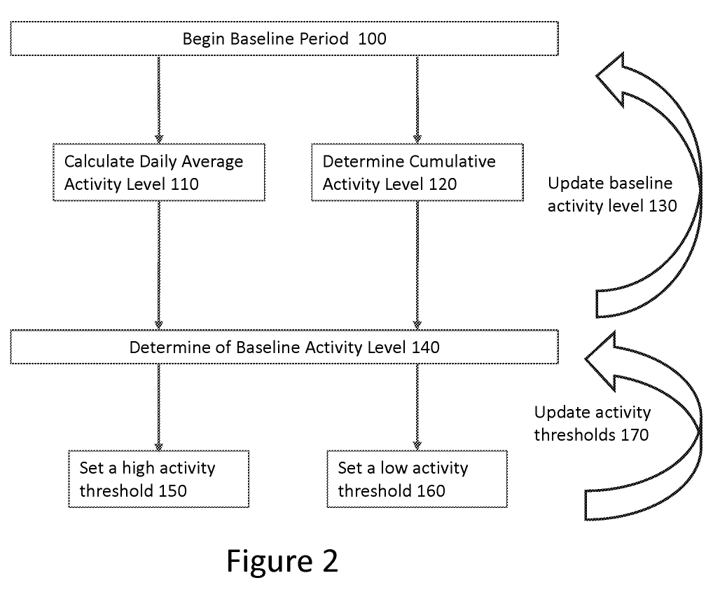

FIG. 2 is a method block diagram illustrating the process of setting of a baseline activity level of the patient and setting a high activity threshold and a low activity threshold at an initial programming session.

The system establishes and determines a baseline activity level of the patient through a monitoring process during a baseline period as indicated at 100. In some examples, the system may monitor a patient's activity level over a period of at least one week to establish a baseline activity level; monitoring the patient’s activity level may occur for as much as five to ten weeks. During this baseline period, activity data may be collected from various sources such as those as described in FIG. 1. For example, movement, galvanic skin response, temperature, heart rate, breathing rate, blood analyte levels, tension, gait, posture, etc. may be monitored using commercial devices (smartphones, “smart” watches, fitness trackers, other wearable devices) and/or medical or special use devices. Thus, the activity data may include various physiological metrics including movement, heart rate, sweat production, oxygen levels, and respiration rates, among others, to provide a comprehensive view of patient activity level. Any one, or combination of multiple, of these metrics may be used in the following examples as a measure of activity level. Still further, one metric may be used to check another metric; for example, output of a movement tracker indicating patient motion can be referenced against a heart rate monitor to confirm the patient is engaged in physical activity, rather than the movement tracker being fooled by noise.

To develop a meaningful pattern of patient activity levels to determine the baseline activity level, the system may utilize a patient's daily average activity level 110 and/or a cumulative activity level 120.

The daily average activity level 110 is calculated following the baseline monitoring period. This average may be assigned to both complete days as well as to consecutive or rolling time windows throughout the day. Rather than relying on instantaneous measurements, a calculation of daily average activity level ensures that the baseline activity level is indicative of the patient's overall condition, rather than a response to brief or temporary fluctuations.

The cumulative activity level 120 provides a summary of activity data over the baseline monitoring period 100. The cumulative activity may be reset periodically, for example, daily. The cumulative activity level may be measured by, for example, units of energy used or work performed, over a time period or window of, for example, at least one hour, such as, without limitation, one to eight hours, or two to six hours, or, in some examples, four hours. For example, the cumulative activity level may be determined by the number of calories burned by walking throughout each afternoon from 12 p.m. to 4 p.m. during the one week baseline monitoring period. The cumulative activity level allows the system to identify shorter-term variations in activity levels that may require therapeutic intervention without reliance on instantaneous measures of patient activity level.

These measures 110, 120 target different things. The cumulative activity level 120 may monitor over a first period of time measured in the range of about 0.5 to about 6 hours, or more or less, to identify cumulative and/or sustained activity in a block. The daily average activity level 110 instead looks at a second, relatively longer period of time to observe how the patient is doing throughout the day at a somewhat higher level. A patient may engage in activity throughout the day, at low levels, leading to a relatively high daily average activity level, which would indicate the patient is receiving adequate therapy and feels well. On the other hand, a patient may engage in a larger amount of sustained activity, such as walking for an hour, yielding a high cumulative activity level 120 even if the period of sustained activity is followed by a long period of rest resulting in an average daily activity level 110 that is closer to baseline, for example. While both measures 110, 120 are shown in this example, one or the other may be omitted if desired.

To ensure continued accuracy of the system, the baseline activity level is periodically recalculated as indicated at 130 in the same manner as the initial baseline activity level. In some examples, the baseline activity level may be updated by tracking patient activity levels over a longer time period, such as monthly or quarterly intervals. By periodically recalculating the baseline activity level, the system may detect sustained changes in the patient's activity levels as treatment progresses. Additionally, periodically recalculating the baseline activity level may account for changes in activity as the patient ages or the patient's condition changes.

Using one or both measures the baseline activity level is determined at 140. After establishing the baseline activity level, the system may then set a high activity threshold 150 and a low activity threshold 160 (collectively referred to as “activity thresholds”) during initial programming. These thresholds may create distinct activity ranges that may trigger a therapeutic response.

The activity thresholds are set relative to the baseline activity level by using the baseline activity level as a reference point. Accordingly, if the baseline activity level is recalculated and/or updated, the activity thresholds are also updated 170 to reflect continued accuracy of the system. The high activity thresholds and low activity thresholds may also be modified at a later period beyond initial programming in response to monitored effectiveness of previous therapeutic interventions, discussed in greater detail below. Other bases for updating the activity thresholds are also contemplated. Regardless of the basis for updating the thresholds, the system may automatically shift the activity thresholds. In other examples, the high activity threshold and the low activity thresholds may be manually set and/or updated and entered into the system by a user.

In some examples, setting the activity thresholds may occur over a predetermined period determined by a clinician, for example, five weeks, until the thresholds have reached a confidence level acceptable to the clinician.

In some examples, there may be up to four or more activity thresholds. For example, a high and low threshold may be set for each of the daily average activity level 110 and the cumulative activity level. This can result in several comparisons to thresholds throughout the day. For example, assuming a four hour period for the cumulative activity level 120, using a rolling window that sums detected patient activity using a first-in-first-out method, the high and low cumulative activity thresholds can be set and compared to the monitored cumulative activity. Likewise, the daily average activity level 110 can also be compared to high and low thresholds. Alternatively, the cumulative activity level 120 can be used to flag periods of sustained activity and thus compared only to a high activity threshold, while the average daily activity is compared to each of high and low thresholds, for example. Various combinations can be used.

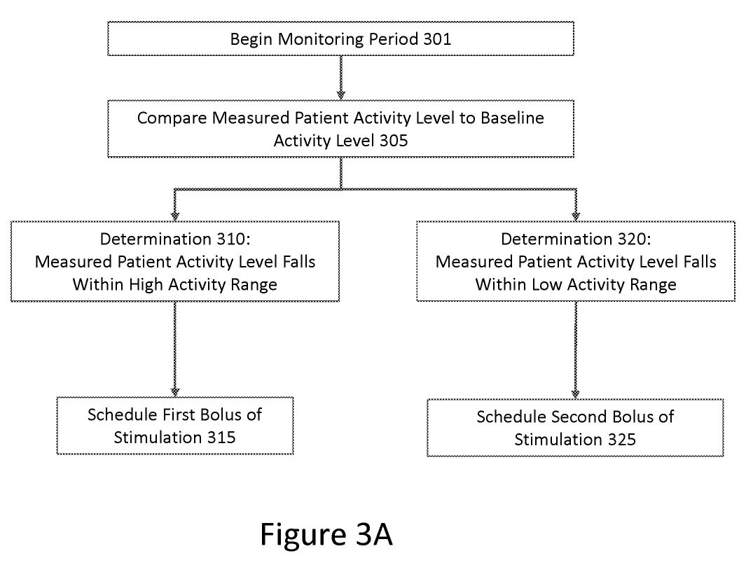

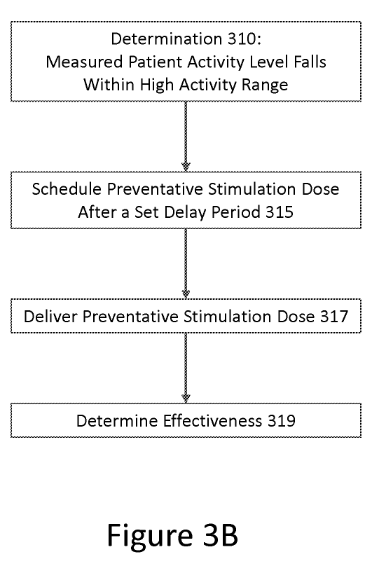

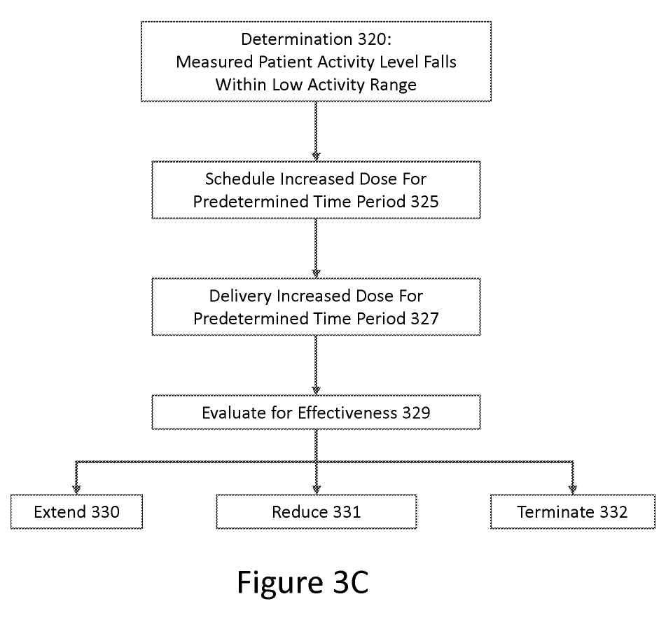

FIG. 3A is a method block diagram illustrating the determination and response to whether a measured patient activity level falls within a high activity range or a low activity range (collectively referred to as the “activity ranges”). This method can apply to either or both of the cumulative activity level or the daily average activity level, as desired. The high activity threshold and the low activity threshold define the high activity range and the low activity range, respectively. These ranges may be used to indicate a need for therapeutic intervention.

The high activity threshold sets the minimum patient activity level to define the high activity range. If the patient activity level is determined to be within the high activity range (in other words, above the high activity threshold), represented by block 310, this may represent a significant increase in the patient activity level throughout an extended period of intense activity, which may be indicative of pain that the patient may experience later due to such high activity. In response, the system may schedule 315 a preventative stimulation dose after a set delay period.

The low activity threshold sets the minimum patient activity level to define the low activity range. If the patient activity level is determined to be within the low activity range (in other words, below a low activity threshold), represented by block 320, this may represent that the patient is spending more time in rest and/or immobile due to experiencing an increased level of pain. In response, the system may immediately schedule 325 a stimulation dose for a predetermined period, typically, but not limited to, four hours.

In some examples, patient activity levels above the low activity threshold, or below the high activity threshold, trigger no therapeutic response and/or no change in the ongoing and in-use therapy regimen. In other words, patient activity levels between the low activity threshold and high activity threshold will not trigger a therapeutic intervention from the method. Likewise, if a single-sided analysis is used, an activity level which does not exceed the high or low activity threshold (whichever is in use) will not trigger a change in the in-use therapy regimen.

In some examples, whether the patient activity level falls within the high activity range or the low activity range is determined by monitoring the activity level of the patient over a monitoring period 301, utilizing the cumulative activity levels associated with the baseline activity level for comparison 305. Other examples may monitor the average daily activity level and trigger a therapeutic intervention using the high and/or low threshold for the average daily activity level metric; the analysis can be triggered daily, such as during a period of rest (overnight, for example).

During the monitoring period, the activity level of the patient is monitored over a predefined time, typically, but not exclusively, four hours, when monitoring the cumulative patient activity level.

Similar to the monitoring of the patient activity levels during the baseline period, measurements of the patient activity levels may be sourced from a variety or a combination of activity sensors such as heart rate monitors, a cardio fitness monitoring/tracking smartphone applications, accelerometer readings, and patient interaction data from the RC.

When monitoring for measured patient activity levels, the system may evaluate (a) whether there are measured patient activity levels within one of the activity ranges at some point during the waking hours of the patient throughout a day and/or (b) whether a measured activity level falls within one of the activity ranges over a set number of consecutive windows or rolling windows. If either evaluation is made, then the system activates the therapeutic response of a limited pain bolus program.

The monitoring periods reoccur periodically, even after the measured patient activity levels are found to be within an activity range. In some examples, if no measured patient activity level falls within either activity range, the system may wait until a subsequent monitoring period to perform the evaluation again. For example, a daily activity monitoring period can restart each day at a predetermined time, or in response to a predetermined condition (patient asleep and/or asleep for at least a set period of time). A cumulative activity monitoring period can be restarted at intervals, if desired, such as, for example, restarting the analysis every 30 or 60 minutes with a look back to a longer window of time such as 2 to 4 hours, so that overlapping windows are set, and/or using a rolling window of time which takes in new activity data on a first-in, first-our basis. Non-overlapping windows for the cumulative activity monitoring period can be used if desired.

In response to the determination 310 that the measured patient activity level falls within the high activity range, the system may automatically schedule 315 and deliver 317 a first bolus of stimulation after a set delay period in accordance with the process illustrated in FIG. 3B.

Patient activity falling within the high activity range may be indicative that the patient is currently being well treated but may experience higher level of pain later, for example, later in the day or the following day, due to the increase in patient activity levels relative to the baseline activity level. Because the pain is expected at a later time, the system may issue a first response of a preventative pain bolus (alternatively referred to as a preventative stimulation dose), scheduled after the set delay period 315.

The set delay period may vary. For example, the set delay period may be predetermined. In some examples, the set delay period may vary based on, but not limited to, physiological responses to intense physical activity. For example, the set delay period may be eight hours after determination that the measured patient activity level falls within the high activity range, reflecting how pain typically manifests after strenuous activity.

However, in some examples, the delay period may be dynamically adjusted. For example, if the set delay is predicted to be within the patient's sleep time, which can be determined by analyzing patterns of low activity levels and/or minimal remote control interaction during specific times of day, the pain bolus may be delivered thirty minutes to an hour before the patient is expected to be awake, regardless of whether the delivery occurs before or after the set delay period. A calendar of patient sleep times may be stored, for example, to determine whether a delay period is needed and how long a delay period ought to be.

In some examples, the delay period may be dynamically adjusted in response to monitoring an effectiveness of the first bolus of stimulation. The monitored effectiveness of the preventative stimulation dose may comprise monitoring changes in measured patient activity levels following the preventative stimulation dose and determining whether the changes in measured patient activity level indicate a return to the baseline activity level established during the baseline period. For example, a significant decrease in measured patient activity levels will shorten the set delay, activating the preventative stimulation dose earlier. In another example, a significant increase in measured patient activity levels may also shorten the set delay to provide more immediate therapeutic intervention. In these examples, a significant decrease/increase may be characterized by a drop/or surge in activity levels compared to the expected average for that time window, with significance being determined by a set percent deviation from the average consistently over a specified period of time. The expected average may be derived from previously measured patient activity levels or average activity levels corresponding to that time window.

The preventative stimulation dose may be delivered at a modified intensity relative to a pain bolus. A pain bolus may deliver a stimulation program to a first location using a first intensity (combination of pulse width, frequency, and amplitude) for a first duration, and the preventative stimulation dose is delivered 317 with at least one of a different location, intensity or duration. For example, a preventive stimulation dose may use a lower amplitude and/or shorter duration than a pain bolus. In some examples a preventive stimulation dose may be issued below a paresthesia threshold, while the pain dose is above the paresthesia threshold, or both may be above or below the paresthesia threshold.

Following the set delay period and delivery 317 of the stimulation dose, the preventative stimulation dose may also be modified based on the effectiveness of the stimulation. To determine effectiveness 319, the system may monitor changes in measured patient activity levels following the second bolus of stimulation and determine whether the changes in measured patient activity level indicate a return to the baseline activity level. If the effectiveness of the stimulation dose is found to be sufficient, modification of preventative stimulation dose may not be warranted. If the effectiveness of the stimulation dose is found to be insufficient, further modification to the intensity of the preventative stimulation dose may be warranted.

In response to the determination 320 that the measured patient activity level falls within the low activity range, the system may automatically schedule 325 and deliver 327 a second bolus of stimulation in accordance with the process illustrated in FIG. 3C. Here, catient activity levels falling within the low activity range may be indicative that the patient may be currently experiencing an increased level of pain. Because the pain may currently exist, the system may issue a second response of issuing an increased stimulation dose for a first predetermined time period, typically a minimum time of four hours. Such increasing dose may be a single step change, or may include a series of changes over the course of a single therapy session, or a series of changes over the course of several therapy sessions, such as by increasing therapy pulse width or amplitude, or number of pulses per session, for example, in several steps taken over the course of several scheduled therapy sessions.

In comparison to the preventative stimulation dose of the first bolus of stimulation, the second bolus of stimulation may be configured with a shorter duration window but at a higher stimulation dose. The higher dose of the second bolus reflects the immediate need to address existing pain conditions, rather than preventing future pain.

The system actively monitors the patient's response to the second bolus of stimulation and evaluates 329 the monitored effectiveness of the patient activity level near end of the first predetermined period, typically the last one or two hours of the predetermined period.

Similar to the first bolus of stimulation, a monitored effectiveness the second bolus of stimulation may comprise monitoring changes in measured patient activity levels following the second bolus of stimulation and determining whether the changes in measured patient activity level indicate a return to the baseline activity level. Dependent on the monitored effectiveness following the second bolus of stimulation, the system may implement an automated response, the responses including extension 330, reduction 331, or termination 332 of the increased stimulation dose.

In response to an instance wherein the evaluated patient activity level remaining unchanged from the measured activity levels prior to the second bolus of stimulation, the increased stimulation dose may be extended 330 for a second predetermined time period. The evaluation process and determination of whether to further extend the increased stimulation dose, reduce the extended increased stimulation dose, or terminate the extended stimulation dose begins, following the same process as the second bolus of stimulation during the first predetermined period, at the end of the second time predetermined period.

In response to an instance wherein the evaluated patient activity level indicates an increase in activity, but that has not yet reached the baseline activity level, the stimulation dose may be reduced 331 for a third predetermined time period, rather than fully resetting to the stimulation parameters prior to the delivery of the second bolus of stimulation. In some examples, the third predetermined time period is less than the first predetermined time period, for example the first predetermined time period may be four hours and the third predetermined time period may be two hours. Relative to the stimulation parameters in place before the delivery of the second bolus of stimulation, the reduced stimulation dose still remains at an increased dosage. The evaluation process and determination of whether to extend the reduced stimulation dose, further reduce the reduced stimulation dose, or terminate the reduced stimulation dose begins at the end of the third predetermined time period, following the same process as the second bolus of stimulation during the first predetermined period.

In other examples, if the reduced stimulation dose leads to an instance wherein the patient activity level further decreases, the system may terminate the reduced stimulation dose and return to the increased stimulation dose of the first predetermined time period that showed an increase in activity for either the same amount of time as the first predetermined time period, for example, four hours, or for a greater time period, for example, eight hours.

In response to an instance wherein the evaluated patient activity levels have significantly increased, that is whether the patient has returned to the baseline activity level, the system may terminate 332 the increased stimulation dose, returning to the stimulation parameters in place prior to the delivery 327 of the second bolus of stimulation.

In response to an instance wherein the evaluated patient activity level further decreases, the system may terminate 332 the increased stimulation dose, returning to the stimulation parameters in place before the delivery of the second bolus of stimulation. In some examples, the system may prompt an additional request for information.

It should be noted that the first, second, and third predetermined time periods may differ from one another, may partially match, or may all be the same; for example, the first predetermined time period may be four hours, the second predetermined time period may be four hours, and the third predetermined time period of two hours.

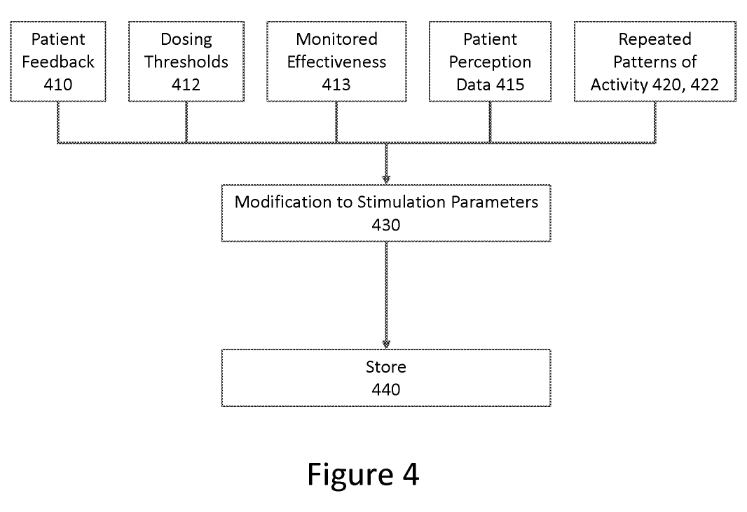

FIG. 4 illustrates bases for modifications to stimulation parameters. Modifications to stimulation parameters may be based on the monitored effectiveness 413 of the first or second stimulation bolus as described above. In some examples, modifications to stimulation parameters may also be based on direct patient feedback 410 to the first or second stimulation bolus or patient perception data 415 collected during initial programming.

In some examples, the patient may provide direct feedback 410 in response to the first or second stimulation bolus through the RC. Feedback may include, but is not limited to, pain ratings on a numeric scale, symptom reporting, patient perception of the effectiveness of the stimulation bolus, responses to inquiries of difficulty or ease of activities following the received stimulation bolus, locations where the patient has experienced the best symptomatic relief.

In response to patient feedback 410, the system may automatically modify 430 the stimulation duration and the stimulation dose level of a subsequent bolus to better achieve improvement in pain levels of the patient. For example, the system may utilize patient feedback to monitor whether the delivered stimulation parameters are achieving the desired therapeutic results. In another example, if the patient provides positive feedback, the system may maintain the stimulation parameters of the first or second stimulation boluses on an ongoing basis – effectively re-calibrating the system.

During initial programming, the user, such as a physician, may establish a maximum stimulation threshold and a minimum stimulation threshold 412 (herein referred to collectively as the “dosing thresholds”) which may define the allowable range for patient control through the patient device. In some examples, the system may set multiple dosing thresholds in order to allow for modifications 430 of the stimulation parameters of the preventative stimulation dose or the increased stimulation dose when the parameters within the allowable range do not result in improvement, such as lack of the monitored effectiveness or lack of positive change in patient feedback.

In some examples, the system may recommend the dosing thresholds based on program-suggested stimulation parameters, tested stimulation parameters, and additional collected perception information collected during the initial programming session. In some examples, the system recommendation may be automatic without prompting the user or requiring user input. In other examples, the system may require that the user initiate a command to the system to provide the recommended stimulation thresholds.

The system may continuously learn from the variety of possible modifications to the stimulation parameters, dosing thresholds, and responses to evaluated patient activity levels to apply these modifications to subsequent boluses of stimulation.

In some examples, the system may also adapt and store 440 modifications in response to repeated patterns of measured activity levels 420, 422 falling within the high activity or low activity range. Repeated patterns 420, 422 may include many instances of falling within one of the ranges, despite updates in the baseline activity level.

In response to repeated patterns of high activity patient levels 420, the system may increase the set delay period to confirm the necessity of the preventative stimulation dose.

In response to repeated patterns of low activity patient levels 422, the system may prompt a troubleshooting session to identify whether the dose is incorrect or inappropriate. The troubleshooting session may also be utilized to determine whether the location needing of treatment may have moved or was previously incorrect.

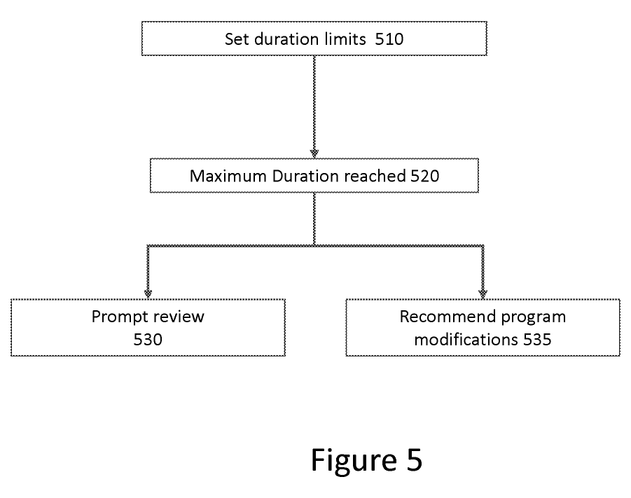

In some examples, the system may be configured to set maximum duration limits 510 on automated adjustments based on monitored effectiveness or observed patient responses to maintain safeguards against excessive consecutive changes, as illustrated in FIG. 5. The maximum duration limit serves to prevent overuse of stimulation, which may lead patient tolerance. As tolerance develops, higher frequency or intensity of stimulation may be needed to compensate for decreased effectiveness, further accelerating the development of tolerance and degradation of long-term effectiveness.

When the maximum duration limit is reached 520 and the desired therapeutic outcome has not been reached, the system may trigger clinical review 530, rather than continuing with automated adjustments. This may create a checkpoint for evaluating effectiveness, prompting a troubleshooting programming session with the patient, and considering alternative programs or stimulation parameters, scheduling changes. In this manner, the maximum duration limit serves to ensure therapeutic outcomes while maintaining appropriate clinic oversight.

In some examples, in addition to prompting review, the system may also recommend modifications 535 to the pain bolus program based on the monitored effectiveness of the first or second bolus of stimulation in a similar manner to the automated responses following the first or second bolus of stimulation. That is, the system recommends program modifications based on whether the delivered stimulation is achieving the desired therapeutic result. In some examples, the system may also base recommended stimulation parameter modifications based on patient feedback. For example, the system may also consider what is tolerable to the patient by incorporation of patient feedback.

In some examples, the system may incorporate expected activity metrics to identify potential instances in which an activity threshold is exceeded when such activity is deemed likely or expected. For example, with the low activity threshold, the system may identify, through monitoring over time, or through patient interaction, that certain time periods are associated with the patient being at rest. For example, the patient may wear a sleep monitoring device (fitness watch, smart watch), or use a sleep aid device (continuous positive air pressure system for sleep apnea) that monitors patient sleep patterns, with which the implanted device or an external patient remote control (or any other suitable system) can communicate, so that the device can be informed of the patient being asleep. In another example, the patient may be asked to input to a patient remote control or other device (such as in response to an email, medical practitioner query, etc.) to indicate times of day that the patient goes to bed/sleep and wakes. In another example, the device may instead infer patient sleep from use (more particularly, lack of use) of the patient’s smartphone, or by identification of repeating patterns of sensed activity from day to day in which patient activity is relatively lower and/or the patient is identified, using for example a postural sensor, as being in a lying down position. In another example, if the patient has a low activity period that crosses the low activity threshold, the implanted device may prompt the patient remote to query the patient (directly or through another device such as the patient’s smartphone, or a home smart device (for example, a smart home device such as an Alexa® device) to confirm whether the patient was asleep and/or whether the patient slept “well” as subjectively understood by the patient; such a query may be posed during a delay period between identifying a potential low activity threshold crossing and actual therapy bolus delivery. It will be recognized that if the patient sleeping deeply without tossing and turning, this likely indicates therapy that is working well, rather than a problem requiring intervention. Thus the system may be configured to identify that the patient is or has been asleep when a low activity threshold is crossed and, if so, to cancel the therapy.

In another example, the low activity threshold and/or high activity threshold may have daily variations. For example, the device may be configured as previously described to identify patterns of sleep and awake. Low activity and/or high activity thresholds can be set separately for each of waking and sleeping hours.

The inclusion of scheduled therapy for the patient, as well as possible scheduling of one or more boluses of therapy in response to high activity threshold or low activity threshold being crossed presents a chance for overlapping therapy requests. A hierarchy can be set.

In one example, referring back to FIG. 3A, the patient’s device operates in accordance with a medically set schedule. If an activity-driven bolus of activity is scheduled by only one of 315 or 325, the activity driven bolus takes precedence, and will occur at its schedule time, pushing the medically set schedule back in time and/or canceling therapy delivery according to the medically set schedule, if the medically set schedule calls for therapy within a preset time period of the bolus, such as within 30 minutes before or after the bolus (or more or less), or during any delay from determination at 310/320, respectively, and the bolus at 315/325, plus a preset time afterwards (5 minutes to an hour, for example). In another example, if each of a high activity range threshold crossing is identified at 310, and a low activity threshold crossing occurs at 320 (which may occur during the delay period from activity to first bolus in accordance with the schedule form 315), the second bolus, based on low activity threshold, is executed and the first bolus is not.

The order of precedence can be modified in other examples. In an example, the patient’s normally scheduled therapy takes precedence, and any overlapping bolus (for example, scheduled to occur within 5 to 60 minute before or after the scheduled therapy, is delayed until a preset period after the normally scheduled therapy is delivered.

In another example, normally scheduled therapy takes precedence over therapy scheduled due to high activity threshold crossing, while delivery of therapy due to low activity threshold crossing takes the highest precedence, delaying the normally scheduled therapy and cancelling therapy in response to high activity threshold crossing.

In another example, therapy in response to high activity threshold crossing takes precedence over normally scheduled therapy, which in turn takes precedence over therapy scheduled due to low activity threshold crossing. Here, all three may be delivered. Alternatively, the existence of a call for therapy due to high or low activity threshold crossing may cancel an instance of the normally scheduled therapy.

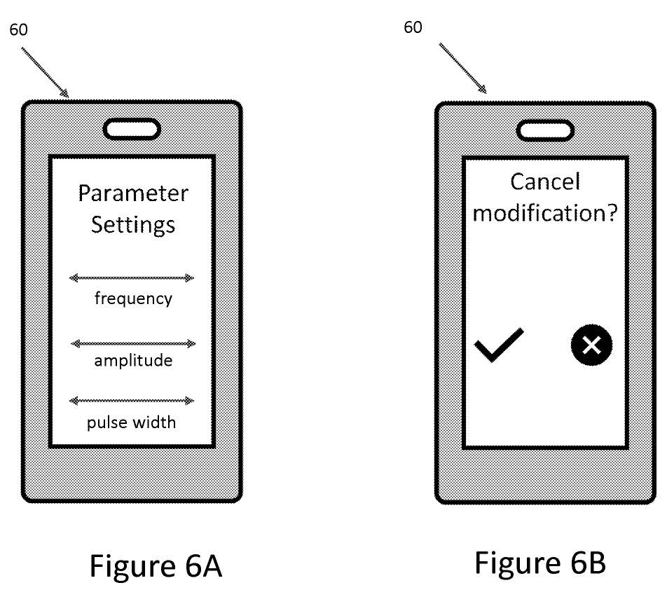

FIGS. 6A-6B show an illustrative patient notification system 60. The system may be configured to notify the patient of any automatic stimulation parameter adjustments. The notifications may be delivered to and displayed on at least one device, including the RC with an integrated user interface, a patient's smartphone (either via text/SMS messages or an installed smartphone application), or other computing devices connected to the system via a Bluetooth connection or other communication network.

Alternatively, the patient may be notified of an automatic stimulation parameter adjustment through a wearable device using discrete vibrations or display messages. In some examples, the wearable device may also include the activity sensor, such as a smartwatch with the cardio fitness tracker application and a separate notification system.

In some examples, the patient notification system may include a patient override interface to prevent automatic implementation of forthcoming or future stimulation parameter adjustments. The patient override interface may also present options to manually modify automatic parameter adjustments within the range defined by the dosing thresholds along with the notification of an upcoming stimulation parameter adjustment, as shown in FIG. 6A.

The patient notification system may include additional safeguards to prevent accidental modification or cancellation to automatic stimulation parameter adjustments. In some examples, the patient notification system may be configured to provide a confirmation screen before implementing any changes deviating from the automatic stimulation parameter adjustment. For example, and as illustrated in FIG. 6B, the patient notification system may provide a confirmation screen requiring the patient to select icons to indicate acceptance or rejection of an upcoming or suggested stimulation parameter adjustment.

In some examples, the patient may provide approval for automatic stimulation parameter adjustments in advance, consequently preventing notifications from occurring. This consent may be provided at initial programming or at a later time. This consent may also be cancelled or modified to require patient approval for some or all automatic stimulation parameter adjustments.

In some examples, the system may notify the patient of repeated patterns of high or low activity. For example, the notification system may alert the patient to contact a clinician or other representative in response to a pattern of low patient activity level. In another example, the notification system may alert the patient that a significant increase in patient activity level may lead to increased pain overall.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments that may be referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method for treating a patient having a pain disorder with an implanted spinal cord stimulation system, the method comprising:
monitoring an activity level of the patient during a baseline period;
determining a baseline activity level for the patient over the baseline period;
setting a high activity threshold and a low activity threshold relative to the baseline activity level to define:
a high activity range above the high activity threshold, and
a low activity range below the low activity threshold;
monitoring the activity level of the patient during a monitoring period, wherein the monitoring period is at least one hour;
determining whether a measured patient activity level falls within one of: the high activity range and the low activity range;
scheduling a first bolus of stimulation from the implanted spinal cord stimulation system in response to a determination that the measured patient activity level falls within the high activity range; and
scheduling a second bolus of stimulation from the implanted spinal cord stimulation system in response to a determination that the measured patient activity level falls within the low activity range.

2. The method of claim 1, wherein monitoring the activity level of the patient comprises receiving activity data from at least one of an accelerometer, a cardio fitness monitor, a fitness tracker application, or patient interaction with a remote control device.

3. The method of claim 1, wherein determining the baseline activity level comprises:
calculating a daily average activity level over at least one week; and
determining a cumulative activity level for at least one time window within each day.

4. The method of claim 1, further comprising:
periodically recalculating the baseline activity level based on the measured activity levels; and
adjusting the high activity threshold and low activity threshold based on the recalculated baseline activity level.

5. The method of claim 1, wherein scheduling the first bolus of stimulation further comprises:
scheduling a preventative stimulation dose after a delay period, wherein the delay period is modified based on a detected change in activity level;
delivering the preventative stimulation dose at a modified intensity relative to a pain bolus; and
delivering the preventative stimulation dose for a predetermined duration, wherein the predetermined duration is adjustable in response to the measured patient activity level.

6. The method of claim 1, wherein scheduling the second bolus of stimulation further comprises:
increasing a stimulation dose for a first predetermined time period;
evaluating the activity level of the patient near the end of the first predetermined time period; and
extending, reducing, or terminating the increased stimulation dose based on the evaluated patient activity.

7. The method of claim 1, further comprising:
monitoring changes in measured patient activity level following the first bolus of stimulation and the second bolus of stimulation to determine an effectiveness of each bolus;
determining a modification to stimulation duration and stimulation dose level for each of the first bolus of stimulation and the second bolus of stimulation based on the determined effectiveness of each bolus;
automatically modifying the stimulation duration and the stimulation dose level of a subsequent bolus based on monitored patient responses; and
storing the modified stimulation duration and the modified stimulation dose level for future boluses of stimulation.

8. The method of claim 1, wherein monitoring the activity level of the patient during a monitoring period comprises calculating a daily average activity level during the course of a day.

9. The method of claim 1, wherein monitoring the activity level of the patient during a monitoring period comprises determining a cumulative activity level for at least one time window within each day, the at least one time window being at least one hour long.

10. A method for managing pain therapy via an implanted spinal cord stimulation system, comprising:
determining a baseline activity level by measuring a patient activity level over a baseline period;
measuring an activity level of a patient over defined time periods to determine a cumulative activity level;
setting a first threshold and a second threshold relative to the baseline activity level to define:
a first activity range above the first threshold,
a second activity range below the second threshold;
comparing the cumulative activity level to the first threshold and the second threshold to determine whether the cumulative activity level falls within one of: the high activity range and the low activity range;
performing a first response including scheduling a first bolus of stimulation from the implanted spinal cord stimulation system in response to a determination that the measured patient activity level falls within the high activity range;
performing a second response including scheduling a second bolus of stimulation from the implanted spinal cord stimulation system in response to a determination that the measured patient activity level falls within the low activity range;
monitoring an effectiveness of the first bolus of stimulation or the second bolus of stimulation; and
modifying at least one stimulation parameter based on monitored effectiveness.

11. The method of claim 10, wherein performing the first response comprises:
applying a first delay before initiating the first bolus of stimulation in response to the measured patient activity level exceeding the first threshold, and
delivering the first bolus of stimulation after the first delay;
wherein the first delay is modified based on a detected change in patient activity level.

12. The method of claim 10, wherein performing the second response comprises:
increasing a stimulation dose for a first predetermined time period;
evaluating the patient activity level near the end of the first predetermined time period; and
extending, reducing, or terminating the increased stimulation dose based on the evaluated patient activity level.

13. The method of claim 10, wherein the cumulative activity level is reset at least daily.

14. The method of claim 10, wherein measuring the patient activity level comprises:
tracking at least one of: movement, heart rate, sweat, oxygen level, or respiration;
tracking measured patient activity levels over time; and
determining the cumulative activity level over a time period of at least one hour.

15. The method of claim 10, further comprising:
setting a maximum duration on modifications to the stimulation parameter;
prompting review when the maximum duration is reached; and
recommending program modifications based on the monitored effectiveness.

16. The method of claim 10, wherein monitoring the effectiveness comprises:
monitoring a change in the measured patient activity level following the first bolus of stimulation or the second bolus of stimulation;
determining, based on the monitored change, whether the measured patient activity level returns to the baseline activity level; and
adjusting the stimulation parameter based on the monitored change.

17. The method of claim 10, further comprising:
receiving patient feedback on the first bolus of stimulation or the second bolus of stimulation;
modifying the stimulation parameter based on the patient feedback
storing the modified stimulation parameter for future use.

18. The method of claim 10, further comprising:
setting a maximum stimulation threshold and a minimum stimulation threshold during an initial programming;
automatically recommending stimulation parameters based on perception information collected during the initial programming; and
modifying the stimulation parameters based on collected perception information within the maximum stimulation threshold and the minimum stimulation threshold.

19. The method of claim 10, further comprising:
tracking the measured patient activity level;
updating the baseline activity level, the high-activity threshold, and the low-activity threshold based on a tracked patient activity level; and
adapting the stimulation parameter based on the updated baseline activity level.

20. A system for providing adaptive pain therapy, comprising:
an implantable pulse generator configured to deliver spinal cord stimulation;
at least one activity sensor configured to monitor an activity level of a patient;
a processor configured to:
determine a baseline activity level during a baseline period;
set a high activity threshold and a low activity threshold relative to the baseline activity level;
measure the patient activity level during a monitoring period of at least one hour;
schedule a first bolus of stimulation from the implantable pulse generator in response to the measured activity level exceeding the high activity threshold;
schedule a second bolus of stimulation from the implantable pulse generator in response to the measured activity level falling below the low activity threshold; and
wait until a subsequent monitoring period in response to the measured activity level being above the low activity threshold and below the high activity threshold.
